(19) United States
(12) Patent Application Publication
(10) Pub. No.: US 2009/0117608 A1
(43) Pub. Date: May 7, 2009

(54) APPARATUS FOR ON-SITE MICROBIAL DIAGNOSIS AND DISINFECTION AND METHOD THEREFOR

(76) Inventors: Sylvain Quessy, St-Anne-sur-Sorel (CA); Ann Letellier, Sainte-Hyaen (CA); Serge Auray, North Hatley (CA)

Correspondence Address: LADAS & PARRY LLP, 224 SOUTH MICHIGAN AVENUE, SUITE 1600, CHICAGO, IL, 60604, US

(21) Appl. No.: 11/876,519

(22) Filed: Oct. 22, 2007

Related U.S. Application Data
(60) Provisional application No. 60/852,991, filed on Oct. 20, 2006.

(51) Int. Cl. C12Q 1/22; C12M 1/00

(52) U.S. Cl. 435/31; 435/287.4

(57) ABSTRACT

An apparatus for on-site microbial diagnosis and disinfection comprising a mobile unit for being brought to an infected site in the event of an emergency. The mobile unit comprises at least a first compartment and a second compartment. The first compartment comprises a laboratory for on-site microbial diagnosis of samples of the site. The second compartment comprises a water supply for washing the site and a disinfectant supply for disinfecting the site. A method comprising on-site evaluation of the biological security of a microbiologically contaminated site and of the washing requirements of the site followed by sampling and on-site diagnosis the sample. A disinfection protocol is provided on-site and then applied, the efficacy of the protocol is later evaluated. Bacterium which are competitors to the identified microbiological agent or agents responsible for contamination or infection are then applied to the site.

### CROSS-REFERENCE TO RELATED APPLICATIONS

The present application is based on the priority U.S. provisional application Ser. No. 60/852,991 filed on Oct. 20, 2006 and incorporated herein in its entirety.

### FIELD OF THE INVENTION

The present invention relates to microbial assessment and/or diagnosis of a site as well as disinfection thereof. More specifically but not exclusively, the present invention is concerned with an apparatus and a method for on-site microbial diagnosis and disinfection.

### BACKGROUND OF THE INVENTION

Sanitary supervision, including odor elimination, over livestock farming facilities, agricultural sites and related structures is becoming a greater public concern due to ever-growing production.

Farmers and agriculturalists are seeking better ways of disinfecting farms and agricultural sites as well as methods therefore in order to prevent repeated infection of farm animals, with infectious pathogenic bacteria, while avoiding any hazard development of hazard to livestock, crops or the environment.

Some known methods of disinfecting farming and agricultural sites include spraying a high-concentration of various known disinfectants directly on the livestock farming facilities by means of shower equipment disposed in the livestock farming facilities or by carrying out direct cleaning and disinfection of farming gauges, passages, floor surfaces and animal body surfaces with the use of a hose. Thus, sanitary supervision over livestock farming and agricultural facilities is carried out in order to lower the rate of livestock and crop disease incidence and mortality, thereby enhancing production output, efficiency and public safety.

The art teaches a variety of mobile units that deal with on-site contamination. For example, the art teaches include a pollution cleanup system and method for on-site treatment of polluted water. The system includes a filter media, a pump, a holding tank and valves for operation of the system. Polluted water is passed through the filter media and cleaned. It is then tested for purity. If needed for further cleaning, the water may be recycled through the filter media. Other examples provide a debris cleaning apparatus mounted on a trailer for toxic waste sites. The cleaning apparatus comprises a crane, a cleaning chamber, holding tanks for washing detergents and rinse water, a boiler to heat the detergent and water, pumps, pipes and auxiliary equipment.

The art also provides for a variety of mobile laboratories. For example, the art teaches a mobile intra-operative microscopic diagnosis laboratory capable of analyzing fresh tissue specimen and providing intra-operative consultation. The art also teaches a self-contained mobile laboratory for on-site testing and certification of industrial slings. This mobile laboratory is capable of carrying the heavy pull test equipment required for stress testing industrial slings having up to at least 50 tons of load capacity. The necessary test equipment is mounted on the vehicle of the mobile laboratory. Other known examples include a multi-purpose laboratory room for mobile applications. The room comprises a housing structure having an airtight laboratory unit and an entrance unit, an air-conditioning and exhausting equipment unit to be installed in the upper part of the housing structure and a drain processing equipment unit to be equipped in the airtight laboratory unit.

Apparatus and methods for disinfecting contaminated farms though have particular challenges. A drawback with known methods and equipment for disinfecting contaminated farms and crops (including facilities and equipment) is that intervention and the and the assessment of the efficacy of intervention is not fast enough, given the fact that infectious outbreaks may occur in very short periods of time thereby providing the infectious agent with sufficient time to damage livestock.

### OBJECTS OF THE INVENTION

An object of the present invention is to provide an apparatus for on-site microbial assessment and/or diagnosis, disinfection and prevention.

An object of the present invention is to provide a method for on-site microbial assessment and/or diagnosis, disinfection and prevention.

### SUMMARY OF THE INVENTION

In accordance with an aspect of the present invention, there is provided an apparatus for microbial diagnosis and disinfection of a contaminated or potentially contaminated site, the apparatus comprising: a mobile unit for being brought to the contaminated or potentially contaminated site, the mobile unit comprising: a first compartment comprising a laboratory for on-site microbial diagnosis of samples of the site; and a second compartment comprising a water supply for cleaning the site and a disinfectant supply for disinfecting the site, whereby, the apparatus provides for performing both on-site diagnosis and cleaning and/or disinfection of the site.

In accordance with another aspect of the present invention, there is provided an apparatus for microbial diagnosis and disinfection of a contaminated or potentially contaminated site, the apparatus comprising: a vehicle; a mobile unit mounted to the vehicle for being brought thereby to the contaminated or potentially contaminated site, the mobile unit comprising: a first compartment comprising a laboratory for on-site microbial diagnosis of samples of the site; and a second compartment comprising a water supply for cleaning the site and a disinfectant supply for disinfecting the site, whereby, the apparatus provides for performing both on-site diagnosis and cleaning and/or disinfection of the site.

In accordance with a further aspect of the present invention, there is provided a method comprising: on-site evaluation of the biological security of a microbiologically contaminated site; on-site evaluation of the cleaning requirements of the site; sampling the site; on-site diagnosis and assessment of the site so as to identify the contaminating agent or agents; providing a disinfection protocol on-site depending on the diagnosis; applying the protocol to the site; evaluating the efficacy of the protocol; and applying bacteria to the site that is a competitor to the identified contaminating agent or agents.

In accordance with a non-restrictive illustrative embodiment of the present invention, there is provided a procedure established with a decisional process comprising: on-site evaluation of the biosecurity including water contamination to evaluate a possible source or sources; of evaluation on the image coupled to the surface; on-site evaluation of the cleanliness of the site; sampling the site to evaluate microbial load; on-site evaluation of cleaning, washing, and disinfection protocol requirements of the site and its application; and/or on-site diagnosis and/or assessment of the site so as to identify a specific contaminating agent or agents and/or indicator micro-organism loads including water contamination; providing a disinfection protocol, on-site, depending on the diagnosis; applying the protocol to the site; post evaluation of the efficacy of the protocol; and/or providing and applying a microbial competitor.

The terms “contaminated” and “infected” as well as the terms “contamination” and “infection” are interchangeable. The term “contaminated site” includes without limitation a building, transportation devices and vehicles, agricultural installations, equipments and the like. The term “potentially contaminated site” includes without limitation, any contaminated site, any allegedly contaminated, any site about to be contaminated, any site in danger of being contaminated, any site selected for diagnosis by any competent authority so as to evaluate contamination if any.

Other objects, advantages and features of the present invention will become more apparent upon reading of the following non restrictive description of illustrative embodiments thereof, given by way of example only with reference to the accompanying drawings.

### BRIEF DESCRIPTION OF THE DRAWINGS

In the appended drawings:

FIG. 1 is a perspective view of the trailer of the on-site microbial diagnosis and disinfection apparatus of the invention in accordance with a non-restrictive illustrative embodiment thereof;

FIG. 2 is a perspective rear view of the inside of the trailer of FIG. 1;

FIG. 3 is a schematic top view of the inside of the trailer of FIG. 1;

FIG. 4 is schematic perspective view of the trailer revealing the inside thereof;

FIG. 5 is a perspective view of a changing zone locker of the invention in accordance with a non-restrictive illustrative embodiment thereof;

FIG. 6 is a perspective view of a clean working zone refrigerator of the invention in accordance with a non-restrictive illustrative embodiment thereof;

FIG. 7 is a perspective view of a shelf and counter assembly of the invention in accordance with a non-restrictive illustrative embodiment thereof;

FIG. 8 is a tool/generator enclosure of the invention in accordance with a non-restrictive illustrative embodiment thereof;

FIG. 9 is a flow chart representing certain steps of the method for on-site microbial assessment and/or diagnosis, disinfection and prevention in accordance with a non-restrictive embodiment of the present invention; and

FIG. 10 shows a cross sectional view of a trailer of the on-site microbial diagnosis and disinfection apparatus of the invention in accordance with another non-restrictive illustrative embodiment thereof.

### DETAILED DESCRIPTION OF THE ILLUSTRATIVE EMBODIMENT

Generally stated, the invention is concerned with an apparatus for on-site microbial diagnosis and disinfection. The apparatus comprises a mobile unit for being brought to an infected site. The mobile unit comprising a laboratory for on-site microbial diagnosis of samples of the site. The mobile unit comprises a second compartment comprising a water supply for washing the site and a disinfectant supply for disinfecting the site. Hence, the mobile unit includes a laboratory compartment and a cleaning/disinfecting-station compartment. The invention is also concerned with a method for on-site microbial diagnosis and disinfection. The method allows for providing a cleaning/disinfection protocol after on-site microbial diagnosis; instant cleaning/disinfection after on-site microbial diagnosis; applying a flora of competing bacteria after on-site microbial diagnosis; verifying decontamination efficacy after cleaning/disinfection.

With reference to the appended drawings, non-restrictive embodiments of the apparatus and methods for on-site microbial diagnosis and disinfection will be described herein so as to exemplify the invention and by no means limit the scope thereof.

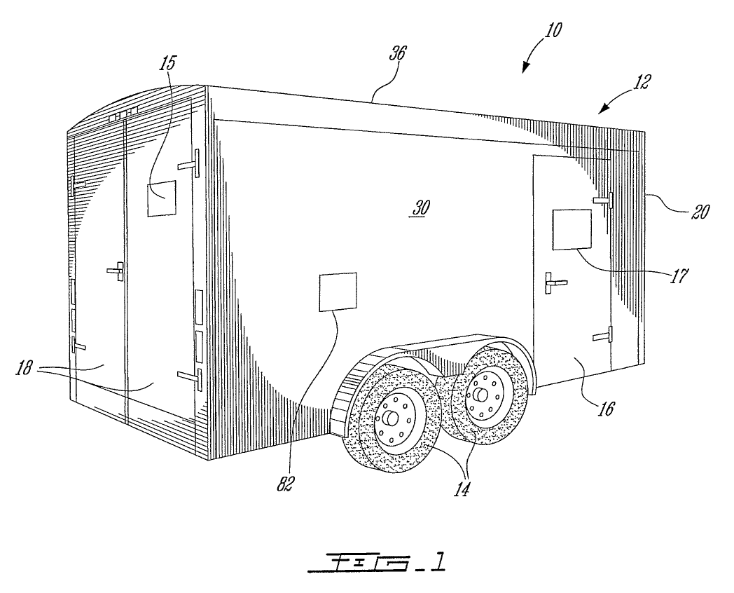

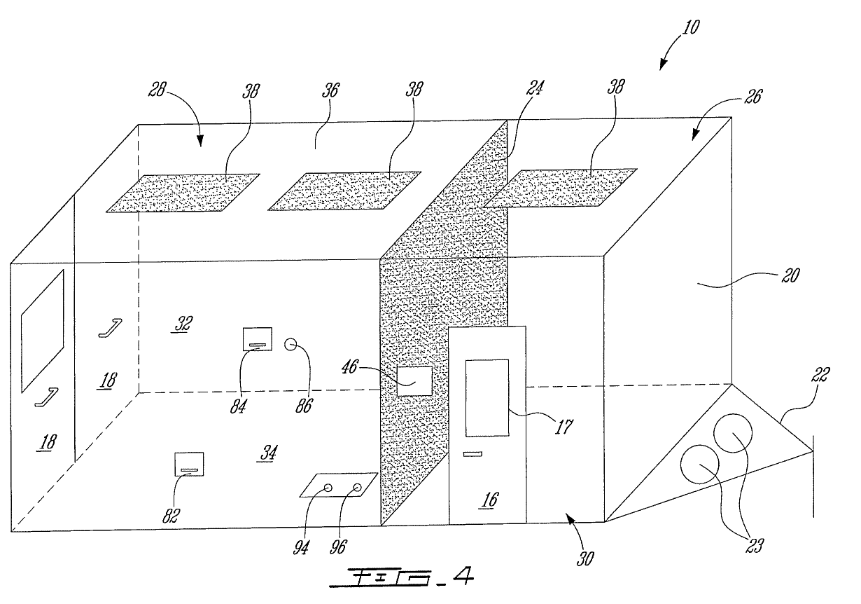

FIG. 1 shows an apparatus 10 for on-site microbial diagnosis and disinfection. The apparatus 10 includes a trailer 12 having wheels 14, a side door 16 with a window 17 (see FIG. 4), back doors 18 and an opposite front side 20. As shown in FIG. 4, the trailer 12 also includes a hitch 22, so as to be pulled by a variety of vehicles (not shown), as well as gas tanks 23.

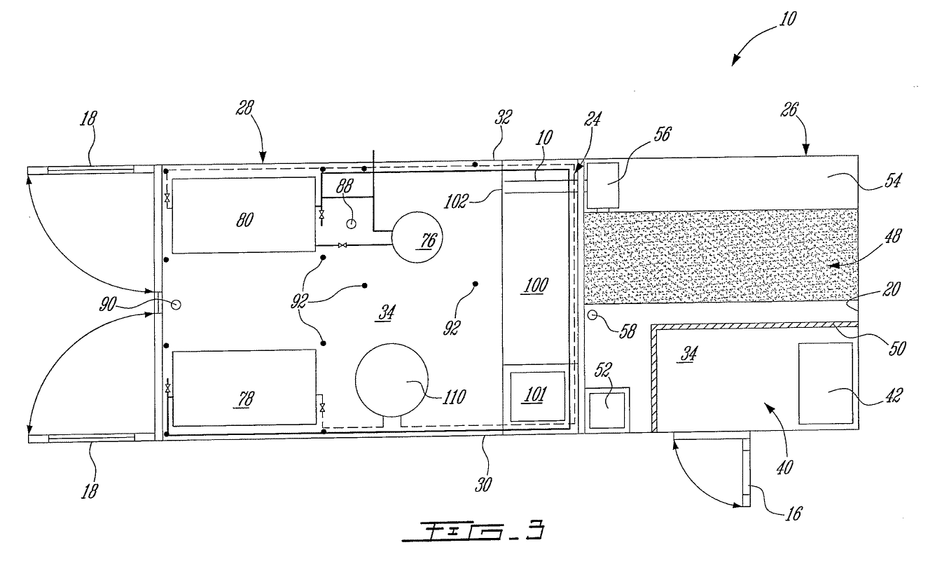

With reference to FIGS. 3 and 4, the inside of the trailer 12 is compartmentalized via a divider wall 24 in at least two adjacent compartments, namely a front laboratory compartment 26 and a back disinfecting-station compartment 28.

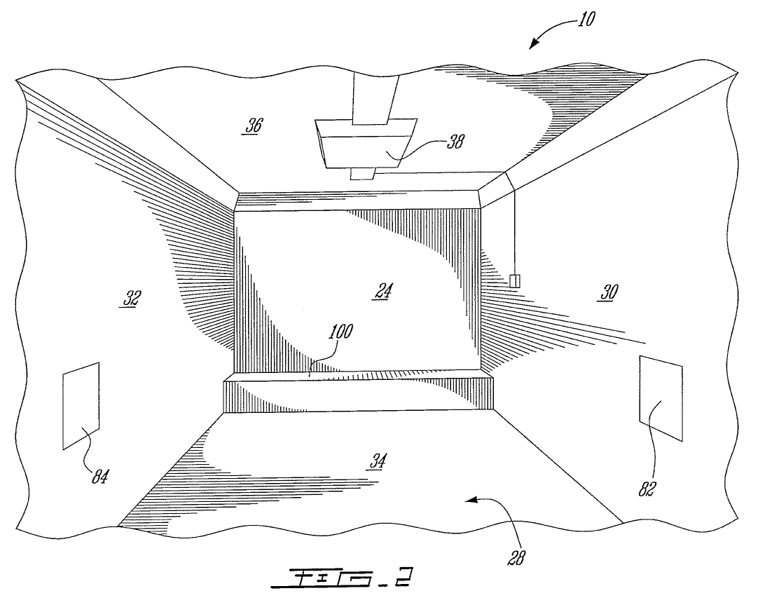

FIG. 2 shows the inside of the disinfecting-station compartment 28, shown to be empty in this case and hence, not including the various components of the apparatus 10.

With reference to FIGS. 1, 2, 3 and 4, the trailer 12 includes side walls 30 and 32, a floor 34, a ceiling 36 with lights 38, a front wall 30 and back doors 18 with a window 15. As will be easily understood by the skilled artisan, the inside of trailer 12 can be compartmentalized in a variety of ways within the context of the invention.

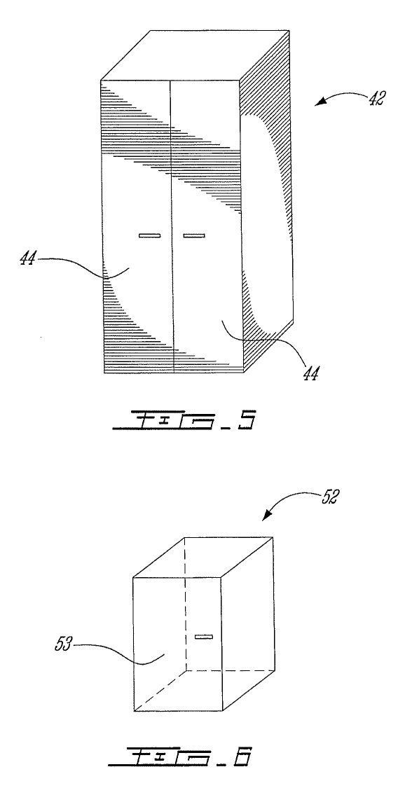

With reference to FIGS. 3 and 4, the laboratory compartment includes an entrance door 16 which accesses a changing zone 40 including a locker 42 also shown in FIG. 5 and including doors 44. A control panel 46 (see FIG. 4) is mounted near door 16 on wall 30 for controlling the temperature and the electrical feed to each of the compartments 26 and 28. The changing zone 40 is separated from a working clean zone 48 via a separator 50. The separator 50 is a thick curtain made out of polyethylene, for example. The clean working zone 48 includes a refrigerator 52 having a door 53 (see FIG. 6), a shelf and counter assembly 54, a heater 56, and a drain 58.

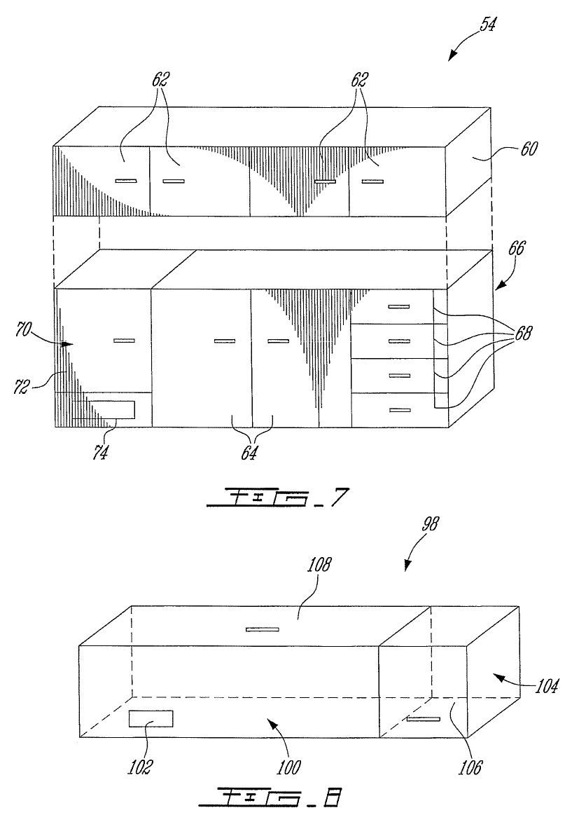

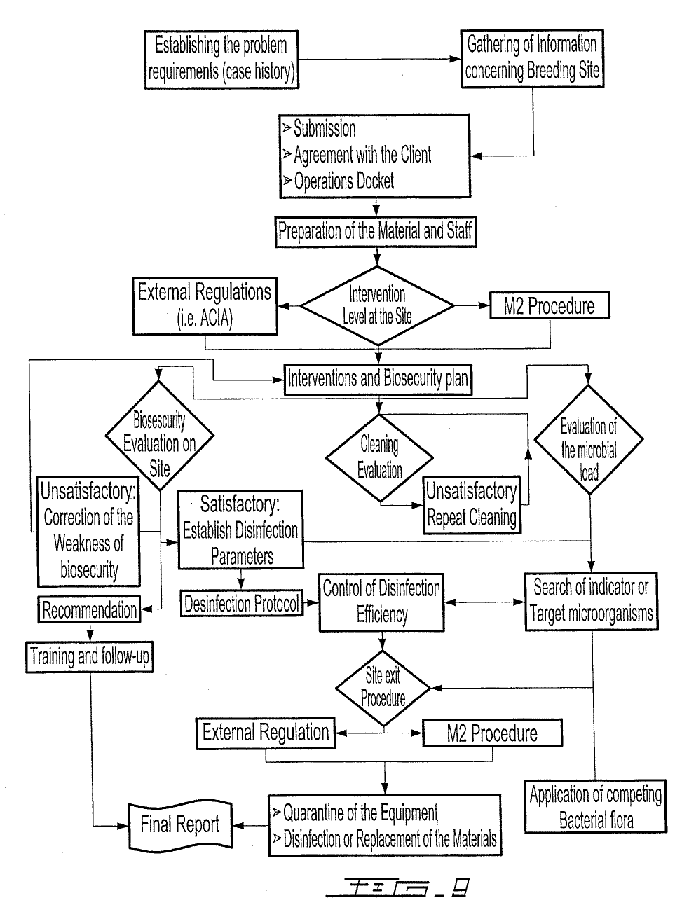

Turning to FIG. 7, the shelf and counter assembly 54 includes a cupboard unit 60 that is mounted on wall 32 and having doors 62; assembly 54 also includes counter 64 having doors 66 and drawers 68. As mentioned, the counter 64 also includes a heating unit 56 (see FIG. 3) within a cabinet space 70 including door 72. A heat exhaust 74 is also provided for heating compartment 26. Doors 62 and 64, 72 and drawers 68 and locker doors 44 are tightly sealed so as to be prevent the contents enclosed therein from being wet during cleaning operations.

Referring now to FIGS. 3 and 4, the disinfection-station compartment 28 includes a water tank 76 that can be brought into fluid communication with either of a pair of compressors 78 and 80 respectively positioned near walls 30 and 32 via conduits (not shown). Each compressor 78 and 80 can be connected to a hose (not shown); a hose roll (not shown) is mounted on either or both walls 30 and 32. The hoses are run through hose-exits 82 or 84 (see FIG. 4) on each side of the trailer 12. A respective pressure-gun (not shown) is mounted to the output aperture of each house providing the user to wash the surrounding area. It should be noted that the trailer 12 also includes a water intake aperture 86 (see FIG. 4) in order to fill the tank 76 with water.

The floor 34 of the disinfection-station compartment 28 includes drains 88 and 90 which can be put into fluid communication with drain 58 via a conduit (not shown) placed on the underside of the floor 34. All water and fluids are recuperated by the trailer 12 so as not to contaminated the site in which it is positioned. The floor 34 of the disinfection-station compartment 28 also includes anchoring members 92 so as to anchor and position various components of the apparatus 10. Furthermore, the floor 34 of the disinfection-station compartment 28 also includes an electrical out-feed 94 and in-feed 96 (see FIG. 4).

With reference to FIGS. 3 and 4, the disinfection-station compartment 28 includes an enclosure 98 including a tool box 100, a generator 101 and a heat exhaust 102 connected to heater 56 via conduit 104 that passes through wall 24 via an opening (not shown) therein. The generator 101 is positioned within a separate compartment 104 which is accessible via a top door 106. The tool box 100 is accessible via door 108. The tool box 100 carries a backpack with a hose and other equipment like a gas-mask with a hose and a handheld sprayer (not shown) as well as a mini-container with a hose and a handheld mist blower (not shown). Doors 106 and 108 are tightly sealed.

The disinfection-station compartment 28 also includes a disinfectant containing tank 110 for containing a variety of appropriate disinfectants or detergents in the form of fluids for example. Of course a plurality of tanks 110 can be included in another non-illustrated embodiment in order to contain various types of disinfectants and/or detergents.

The trailer 12 also includes stabilizers (not shown) on its underside in order to keep it in place when the vehicle which brings it on-site has been removed. These stabilizers can be telescoping legs, scissor jacks or any other extension member as can be contemplated by the skilled artisan.

In operation, the trailer 12 is driven to a site, such as a farm or any other agricultural terrain and the like that requires emergency assistance due to microbiological contamination of any sort. The trailer 12 is installed in an appropriate or convenient area on the contaminated terrain, stable, farm or any like region, area or site. Optionally, the vehicle may be detached from the hitch 22 and the trailer 12 stabilized via stabilizers, such as scissor jacks to give but one non-limiting example. Microbiologists then scout and sample the contaminated site using a variety of known sampling devices. Once the contaminated area has been thoroughly sampled, the microbiologists then enter the laboratory compartment 26 via door 16.

Once in the changing zone 40, they will remove their outdoor attire and put on lab coats, surgical gloves, hair nets and indoor footwear. The outdoor attire is placed within locker 42.

Once changed into their laboratory attire, the microbiologists traverse through the protective curtain 50 into the clean working area 48. The samples are then probed and small amounts of contaminated external material are inoculated and streaked onto Petri dishes or put into tubes containing liquids or gels or other methods to grow, analyze or store microorganisms and processed by appropriate diagnostic devices. This work is performed on the bench lab counter 66 which includes gas outputs for burners, electrical outputs for microscopes and other laboratory supplies used during microbiological diagnostic tests. Inoculated tubes, dishes and the like are then placed in incubators for a predetermined period. Once incubated, the samples are analyzed and the microbial agent or agents identified by further characterization methods or stored in the refrigerator for later analysis.

The equipment used in the laboratory may include without limitation PCR equipment, a bioluminometer, a laminar flow hood, a burner, an incubator, a refrigerator, a freezer, a computer, a printer and other laboratory supplies.

Depending on the concentration and type of microbial agent as well as the infected portions of the contaminated site, the microbiologists then prepare a protocol for the users of the contaminated site (farmers, agriculturists etc.) so as to take the required steps in order to minimize risl future contamination. Contaminated biological waste are sterilized on site. Laboratory clothes and/or clothes used during scouting and sampling the site can be discarded in sealable waste containers within the apparatus 10.

In one non-limiting embodiment, the foregoing is performed in a period of about 1 to 18 hours following sampling.

Concurrently or following this work, technicians in the disinfecting station 28 exit through the back doors 18 and pass hoses out of each side of the trailer 12 via hose exits 82 and 84. In this way, the contaminated site is cleaned (optional) with water and/or detergent with sufficient pressure provided by compressors 78 and 80 as needed. A disinfectant can also be sprayed on the site. Before the disinfection, the technicians dilute the disinfectant in tank 110 into the backpack container or other type of suitable containers or equipment and the disinfectant is spayed as a foam onto the site. After this if need be, the mist blower is used to spray the disinfectant into the air and particle openings of any structure of the contaminated site.

The tank 110 can be used when needed for the storage of disinfectant, detergent or competing flora.

Of course, the skilled artisan will easily understand that a variety of disinfecting protocols and diagnostic methods can be used depending on the type of microbial agent that has contaminated the site, the type of site, the amount of contamination and other variables as is understood in the art.

Thus, the present invention also provides a method including an on-site emergency response of a mobile unit for diagnosis and disinfection. In accordance with this method, the biosecurity of the site is evaluated followed by a cleaning audit. The site is then sampled and diagnosed. Depending on the diagnosis a disinfection protocol is provided. The protocol is applied and the efficacy of decontamination is verified. Finally, a flora of competing bacteria can be applied to the site.

In general, solid waste (clothes for example) are placed in bags which are disinfected with standard chemical sterilization procedures. The trailer apparatus 10 is disinfected with a hose and a suitable disinfectant. The various drains of the invention are in fluid communication within one another as such a pipe can be attached to this fluid circuit in order to recuperate all fluid waste that can be diverted towards a waste fluid reservoir mounted to the apparatus 10.

The apparatus 10 is also useful in emergency cases of infected sites but the invention is not limited to emergency procedures.

The method of the present invention includes the following procedural steps:

1—Evaluation of the problem and the needs of the site; gathering of information regarding livestock breeding, the site etc.

2 —Preparation of materials and training;

3 —Execution of action plan on the site (positioning of apparatus 10, defining the security perimeter, preparing technical assistance etc.);

4 —Evaluation of biosecurity, cleaning, microbial presence;

5—Applying a disinfection protocol adapted to the particular needs of the site;

6—Evaluation of the site after disinfection (possibility of repeating disinfection);

7—Exit procedure: waste treatment, disinfection of apparatus 10; and

8—Reporting to client.

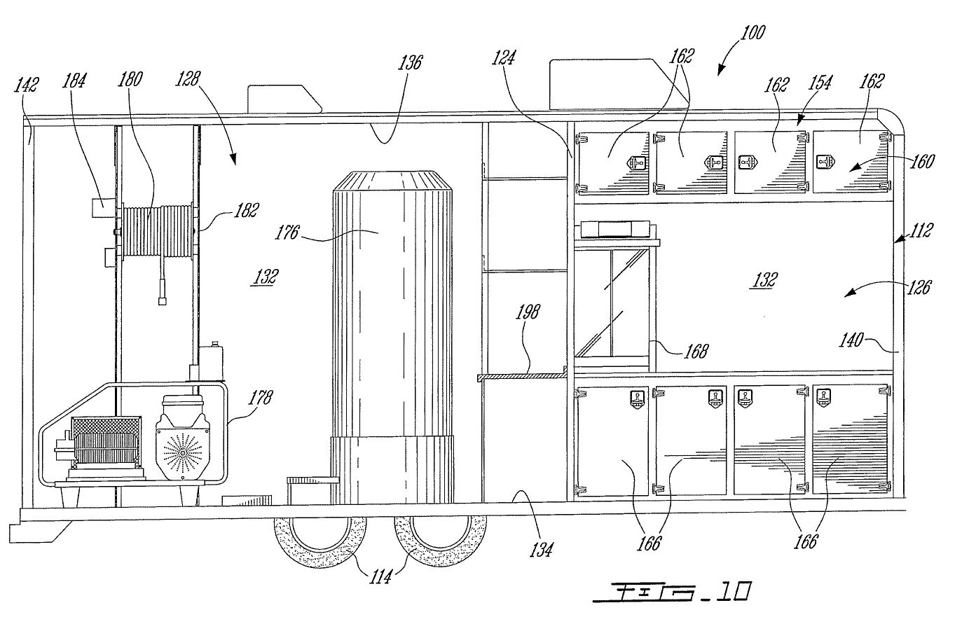

With reference to FIG. 9, a flow chart represents the steps of the intervention procedure in accordance with the present method. The “M2” procedure refers to a variety of possible scenarios that are determined on-site depending on the particular needs thereof. The steps included in the “M2” procedure are: evaluating the biosecurity of the site; evaluating the cleaning procedure of the site; diagnosis; disinfection and disinfection control.

Having now described an illustrative embodiment of the apparatus 10 for on-site microbial diagnosis and disinfection and method therefor as well as the operation thereof, other non-limiting embodiments of the apparatus 10 will now be briefly discussed so as to further exemplify the invention only.

The trailer 12 can be provided in variety of configurations and sizes with wheels 14 positioned in various convenient ways. In this sense, the trailer can be compartmentalized in other ways as can be contemplated by the skilled artisan. To mention only a few examples, the trailer 12 can include two floors, each defining a compartment 26 or 28, the compartments 26 or 28 may be placed side to side along the length of the trailer 12 rather than separated along the width thereof. Of course, in other embodiments, the trailer 12 may comprise physically separated units, each defining a compartment 26 or 28. In still another embodiment, the trailer 12 may include a plurality of compartments 26 and/or 28. In yet another embodiment, the trailer of the invention as well as the compartments may form a single integrates unit with a vehicle.

Compartments 26 and 28 may be configured in various shapes and sizes and internally arranged in various convenient ways. As such, various refrigeration, heating, and/or laboratory equipment can be provided and positioned in a variety of ways known in the art. Various types of compressors for water and/or detergents and/or disinfectant can be used. Different lighting systems can also be provided. In fact, the number, configuration, type, position, size and arrangement of the various components exposed above can be varied within the scope of the invention as someone having skill in the art will immediately comprehend. As such, in the example shown herein, heating is provided via the floor and air conditioning via the ceiling, yet this can be reversed for example.

The components of the invention are removable and replaceable and the various doors and drawers are sealable so as to provide for easily washing the inside of the compartments 26 and 28 without wetting the inside of any enclosures.

The trailer can be made out of a variety of suitable materials. In one non-limiting embodiment, the trailer 12 as well as its walls and counter 66 are made out of a non-corrosive metal.

The separating curtain 50 can be made of a variety of suitable materials; in another non-limiting embodiment, the separator 50 is a wall.

The trailer 12 of the invention is a self sufficient mobile unit comprising at least two compartments. The trailer 12 includes an electrical supply, a gas supply and a water supply. The trailer 12 is also configured to receive external electricity, gas and water when on-site if need be and if the site provides for such.

With reference to FIG. 10, a further non-restrictive illustrative embodiment of the present invention will be described so as to provide an additional non-limiting example only.

FIG. 10 shows a shows an apparatus 100 for on-site microbial diagnosis and disinfection. The apparatus 100 includes a trailer 112 having wheels 114, the inside of the trailer 12 is compartmentalized via a divider wall 124 in at least two adjacent compartments, namely a front laboratory compartment 126 and a back disinfecting-station compartment 128. The trailer 112 includes side walls 130 (only one side wall is shown), a floor 134, a ceiling 136, a front wall 140 and a back wall 142. The laboratory compartment 126 includes a shelf and counter assembly 154 having a cupboard unit 60 that is mounted on wall 132 and having doors 62; assembly 154 also includes counter 164 having doors 166. The laboratory compartment 126 is shown including an incubator 168. The disinfection-station compartment 128 includes a water tank 176 in fluid communication with compressor (pressure washer) 178 connected to a hose 180 mounted on an automatic hose roller 182 powered by motor 184. The disinfection-station compartment 128 includes an enclosure 198 for a tool box 100, a generator, a heater for example or other equipment.

It should be noted that the various components and features of apparatus 10 and 100 described above can be combined in a variety of ways so as to provide other non-illustrated embodiments within the scope of the invention.

It is to be understood that the invention is not limited in its application to the details of construction and parts illustrated in the accompanying drawings and described hereinabove. The invention is capable of other embodiments and of being practiced in various ways. It is also to be understood that the phraseology or terminology used herein is for the purpose of description and not limitation. Hence, although the present invention has been described hereinabove by way of embodiments thereof, it can be modified, without departing from the spirit, scope and nature of the subject invention as described herein.

What is claimed is:

1. An apparatus for microbial diagnosis and disinfection of a contaminated or potentially contaminated site, said apparatus comprising:
a mobile unit for being brought to the contaminated or potentially contaminated site, said mobile unit comprising:
a first compartment comprising a laboratory for on-site microbial diagnosis of samples of the site; and
a second compartment comprising a water supply for cleaning the site and a disinfectant supply for disinfecting the site,
whereby, said apparatus provides for performing both on-site diagnosis and cleaning and/or disinfection of the site.

2. An apparatus according to claim 1, further allowing for providing an on-site protocol for minimizing the risk of future contamination of the site.

3. An apparatus according to claim 1, wherein said mobile unit is mountable to a vehicle.

4. An apparatus according to claim 1, wherein said mobile unit comprises a trailer.

5. An apparatus according to claim 1, wherein said first and second compartment are adjacently disposed in a side by side fashion within said mobile unit.

6. An apparatus according to claim 1, wherein said first and second compartment are separated by a divider wall.

7. An apparatus according to claim 1, wherein each said first and second compartment includes a respective door for allowing access from outside said mobile unit therein.

8. An apparatus according to claim 1, wherein said laboratory compartment comprises a separator thereby providing a changing zone and a separate working zone.

9. An apparatus according to claim 1, wherein said mobile unit comprises a fluid circuit for recuperating all water and fluids used during cleaning and disinfecting thereof, thereby avoiding contaminating the site.

10. An apparatus according to claim 1, wherein said laboratory compartment comprises equipment for on-site disinfection or discarding of laboratory clothes or clothes used during scouting and sampling of the site.

11. An apparatus according to claim 1, further comprising a power generator so as to provide power to said mobile unit.

12. An apparatus according to claim 1, further comprising a heater.

13. An apparatus according to claim 1, wherein said first compartment comprises laboratory equipment.

14. An apparatus according to claim 1, wherein said second compartment comprises a water tank, a disinfectant containing tank, a compressor, and hoses.

15. An apparatus according to claim 1, wherein said mobile unit comprises stabilizers for stabilization thereof on the site.

16. An apparatus for microbial diagnosis and disinfection of a contaminated or potentially contaminated site, said apparatus comprising:
a vehicle;
a mobile unit mounted to said vehicle for being brought thereby to the contaminated or potentially contaminated site, said mobile unit comprising:
a first compartment comprising a laboratory for on-site microbial diagnosis of samples of the site; and
a second compartment comprising a water supply for cleaning the site and a disinfectant supply for disinfecting the site,
whereby, said apparatus provides for performing both on-site diagnosis and cleaning and/or disinfection of the site.

17. An apparatus according to claim 16, further allowing for providing an on-site protocol for minimizing the risk of future contamination of the site.

18. An apparatus according to claim 16, wherein said vehicle and said mobile unit are integrally joined.

19. An apparatus according to claim 16, wherein said vehicle and said mobile are removably joined.

20. A method for on-site microbial diagnosis and disinfection, said method comprising:
on-site evaluation of the biological security of a microbiologically contaminated site;
on-site evaluation of the cleaning requirements of the site;
sampling the site;
on-site diagnosis and assessment of the site so as to identify the contaminating agent or agents;
providing a disinfection protocol on-site depending on the diagnosis;
applying the protocol to the site;
evaluating the efficacy of the protocol; and
applying bacteria to the site that is a competitor to the identified contaminating agent or agents.
